SEQUENCE LISTING (26 sequences)

SEQ ID NO: 1 - protein, 14 aa, Artificial Sequence
  concensus sequence
IEGPTLRQWLAARA

SEQ ID NO: 2 - protein, 5 aa, Artificial Sequence
  binding peptide sequence
APDTP

SEQ ID NO: 3 - protein, 7 aa, Artificial Sequence
  kidney targeting sequence
CLPVASC

SEQ ID NO: 4 - DNA, 1980 bp, Murine leukemia virus
aattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtgggactcctctctcccctacccaacttccaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaaga

SEQ ID NO: 5 - protein, 7 aa, Artificial Sequence
  kidney targeting sequence
CGAREMC

SEQ ID NO: 6 - protein, 9 aa, Artificial Sequence
  brain targeting sequence
CLSSRLDAC

SEQ ID NO: 7 - protein, 21 aa, Artificial Sequence
  brain targeting sequence
WRCVLREGPAGGCAWFNRHRL

SEQ ID NO: 8 - protein, 13 aa, Artificial Sequence
  Synthetically generated peptide
CAAAGRGDSPTRC

SEQ ID NO: 9 - DNA, 39 bp, Artificial Sequence
  Synthetically generated oligonucleotide
tgcgcggccgctggccgtggcgattctcccacgcgttgt

SEQ ID NO: 10 - DNA, 39 bp, Artificial Sequence
  Synthetically generated oligonucleotide
acaacgcgtgggagaatcgccacggccagcggccgcgca

SEQ ID NO: 11 - protein, 21 aa, Artificial Sequence
  Synthetically generated peptide
CAAAQGATFALRGDNPQGTRC

SEQ ID NO: 12 - DNA, 50 bp, Artificial Sequence
  Synthetically generated oligonucleotide
ggccgctcaaggcgcaacgttcgcgctcagaggcgataatccacagggga

SEQ ID NO: 13 - DNA, 50 bp, Artificial Sequence
  Synthetically generated oligonucleotide
cgcgtcccctgtggattatcgcctctgagcgcgaacgttgcgccttgagc

SEQ ID NO: 14 - protein, 6 aa, Artificial Sequence
  Synthetically generated peptide
GRGDSP

SEQ ID NO: 15 - protein, 14 aa, Artificial Sequence
  Synthetically generated peptide
QGATFALRGDNPQG

SEQ ID NO: 16 - DNA, 22 bp, Artificial Sequence
  Synthetically generated oligonucleotide
ttttgtcaagaccgacctgtcc

SEQ ID NO: 17 - DNA, 22 bp, Artificial Sequence
  Synthetically generated oligonucleotide
cgggagcggcgataccgtaaag

SEQ ID NO: 18 - protein, 21 aa, Artificial Sequence
  Synthetically generated peptide
CAAAEQRLGNQWAVGHLMTRC

SEQ ID NO: 19 - DNA, 47 bp, Artificial Sequence
  Synthetically generated oligonucleotide
ggccgagcagcgcctgggcaaccagtgggccgtcggccacctgatga

SEQ ID NO: 20 - DNA, 47 bp, Artificial Sequence
  Synthetically generated oligonucleotide
cgcgtcatcaggtggccgacggcccactggttgcccaggcgctgctc

SEQ ID NO: 21 - DNA, 71 bp, Artificial Sequence
  Synthetically generated oligonucleotide
ggccgcttcacaccttgtaaagtgcgcagagaaggaaaagacgttctgcgtcaacggcgtgagtgttacag

SEQ ID NO: 22 - DNA, 84 bp, Artificial Sequence
  Synthetically generated oligonucleotide
gccgtaggtcttaaccctgtaacactcaccgccgttgacgcagaacgtcttttccttctctgcgcactttacaaggtgtgaagc

SEQ ID NO: 23 - DNA, 83 bp, Artificial Sequence
  Synthetically generated oligonucleotide
ggttaagacctacggctatctgatgtgcaagtgtccgaacgagttcacgggtgaccggtgccagaactacgtcatcgcgtcga

SEQ ID NO: 24 - DNA, 71 bp, Artificial Sequence
  Synthetically generated oligonucleotide
cgcgtcgacgcgatgacgtagttctggcaccggtcacccgtgaactcgttcggacacttgcacatcagata

SEQ ID NO: 25 - protein, 21 aa, Artificial Sequence
  Synthetically generated peptide
CAAAQGATFALRGENPQGTRC

SEQ ID NO: 26 - protein, 56 aa, Artificial Sequence
  Synthetically generated peptide
CAAASHLVKCAEKEKTFCVNGGECYRVKTYGYLMCKCPNEFTGDRCQNYVIASTRC

(19) United States
(12) Patent Application Publication
(10) Pub. No.: US 2005/0221289 A1
(43) Pub. Date: Oct. 6, 2005

(54) Altering viral tropism

(76) Inventors: Michael R Green, Boylston, MA (US); Timothy J. Gollan, Sunnyvale, CA (US)

Correspondence Address: FISH & RICHARDSON PC, P.O. BOX 1022, MINNEAPOLIS, MN, 55440-1022, US

(21) Appl. No.: 10/507,232

(22) PCT Filed: Mar. 7, 2003
(86) PCT No.: PCT/US03/07323

§ 371 Date: Apr. 19, 2005

Related U.S. Application Data
(60) Provisional application No. 60/362,655, filed on Mar. 8, 2002.

(51) Int. Cl. C12Q 1/70; C07H 21/04; C07K 14/15; C12N 7/00; C12N 15/867

(52) U.S. Cl. 435/5; 435/69.3; 435/235.1; 435/325; 435/456; 530/350; 536/23.72

(57) ABSTRACT

Methods of altering retroviral tropism have been discovered. Such methods are useful, e.g., for developing retroviral vectors for gene therapy.

### TECHNICAL FIELD

This invention relates to virology.

### BACKGROUND

Recombinant retroviral vectors are attractive vehicles for gene delivery but they generally lack the cell specificity that is desirable for applications involving gene therapy. For example, the Murine Leukemia Virus (MLV) ecotropic envelope protein (Moloney MLV envelope protein; MoMLV envelope protein) binds to an amino acid transporter that is expressed only in mouse cells and the cells of closely related species (Albritton et al., 1989, Cell 57:659-666), but not in human cells. Host range is determined by regions of variable sequences (termed VRA, VRB) within the extracellular domain (SU) of envelope protein (envelope).

### SUMMARY

The invention is based in part on the discovery that retroviral tropism of ecotropic MLV can be altered or redirected using heterologous short peptide ligands inserted within the retroviral envelope protein of this virus to form chimeric envelope proteins. Such chimeric envelope proteins can be incorporated into a viral vector to create a pseudotyped virus. Wild-type envelope sequence does not have to be deleted for the chimeric envelope proteins to be effective for binding or transduction of a pseudotyped virus that incorporates them nor do they require the presence of an intact wild-type envelope for efficient transduction. In addition, it has been discovered that the length and position of the inserted peptide ligand can affect viral tropism. Thus, the invention relates to a novel method for targeting retroviruses to specific cells by modifying viral envelope proteins. The chimeric envelope proteins are useful, e.g., for creating a vector that can transduce a target cell (for example, a human cell) and for introducing a gene into such a targeted cell, for example, to selectively target and destroy human cancer cells.

In one embodiment, the invention is a recombinant chimeric envelope protein that includes a wild-type ecotropic Murine Leukemia Virus (MLV) envelope protein and a heterologous short peptide ligand inserted within the MLV envelope protein. The invention also includes a nucleic acid sequence encoding such recombinant envelope proteins and plasmid vectors that contain such sequences. The heterologous short peptide ligand can be an RGD ligand, a human epidermal growth factor receptor (HRG) ligand, or a gastrin releasing protein (GRP) ligand. In some aspects of the invention, the heterologous short peptide ligand is flanked by at least one cysteine on each side. In another aspect of the invention, the heterologous short peptide ligand is inserted into a conserved region of a wild-type envelope protein.

The invention also includes a vector comprising a nucleic acid or gene encoding a chimeric envelope protein that contains a heterologous short peptide ligand. The vector can also contain a nucleic acid sequence that codes for a therapeutically useful protein.

In another embodiment, the invention is a recombinant retroviral particle that contains a chimeric envelope protein containing a heterologous short peptide ligand. In some embodiments, such recombinant retroviral particles can infect a mouse cell or a target host cell. In other embodiments, the recombinant retroviral particle cannot infect a mouse cell.

In another aspect, the invention includes a method of altering murine leukemia virus (MLV) retroviral tropism by introducing into the genome of an MLV a nucleic acid sequence that codes for a recombinant envelope protein that codes for a heterologous short peptide ligand. In some embodiments of the invention, the virus cannot express wild-type envelope protein. In another embodiment, the heterologous short peptide ligand is inserted into a conserved region of a wild-type envelope protein.

The invention also includes a method of identifying a chimeric envelope protein that alters viral tropism by introducing into the genome of an MLV a nucleic acid sequence encoding a recombinant envelope protein containing a heterologous short peptide ligand thus making a recombinant virus, infecting a target host cell with the virus, and assaying transduction of the target host cell by the virus, such that transduction of the host cell by the virus indicates that the recombinant envelope protein alters viral tropism. In this method, the heterologous short peptide ligand can be located in a conserved region of the MLV envelope protein, and the target host cell can be a human cell. More specifically, the target host cell can be a cancer cell or a cell that contains a defective gene. In some embodiments, the chimeric envelope protein contains an RGD ligand, an HRG ligand, or a GRP ligand.

In another aspect, the invention includes a method of delivering a gene to a cell by infecting a cell with a virus, e.g., a retrovirus, containing a chimeric envelope protein comprising a heterologous short peptide ligand and a gene. The ligand can be an RGD ligand, an HRG ligand, or a GRP ligand. The host cell can be an animal cell, e.g., a mammalian or human cell, e.g., a cancer cell. Further, the cell can be in an animal, e.g., in a human.

The invention also includes a method of treating cancer by infecting a cancer cell with a virus, e.g., a retrovirus, containing a chimeric envelope protein that includes a heterologous short peptide ligand and a gene that can be used to treat the cancer. The cancer to be treated can be in an animal, such as a mammal, e.g., a human subject. In some aspects, the therapeutically useful gene codes for thymidine kinase.

A “heterologous short peptide ligand” is a peptide between 3 and 90, e.g., 3 and 83, or 6 and 21 amino acids in length, that can specifically bind to a receptor on a cell. The short peptide sequence is heterologous with respect to the wild-type envelope protein into which it is inserted. Examples of heterologous short peptide ligands include RGD ligands, GRP, and HRG ligands as described herein. Other heterologous short peptide ligands can be identified using methods known in the art and the methods described herein.

A “chimeric envelope protein” is a polypeptide containing a retroviral wild-type envelope protein sequence (e.g., an ecotropic MLV envelope protein) into which has been inserted a heterologous short peptide ligand. The chimeric envelope protein may contain the complete sequence of the envelope protein from which it is derived. In some cases a portion (e.g., 1 to about 110 amino acids) of the wild type envelope protein is deleted. A nucleic acid sequence coding for a chimeric envelope protein contains a nucleic acid sequence coding for an envelope protein and a nucleic acid sequence coding for a heterologous short peptide ligand that is inserted in-frame.

A “target host cell” is a cell that can be transduced by a pseudotyped virus containing a chimeric envelope protein. In general, a target host cell is not from the same species as the host cell for the wild-type virus from which the pseudotyped virus is derived. Typically, the pseudotyped virus will bind only to the target host cell and not to other cell types. If the parent virus (i.e., the wild-type virus) used to produce the pseudotyped virus can bind to cells of the host, it is generally desirable to reduce or eliminate this binding, for example, by mutation of the binding site. Host cells can be mammalian, e.g., dog, cat, cow, horse, monkey, or human cells. A host cell can be isolated from a host animal and cultured, or cultured and reintroduced into the host. Alternatively, a host cell can be within the host animal, e.g., in a specific tissue in the host such as muscle, blood progenitor or mature blood cell, liver, kidney, or a tumor or other diseased tissue.

A “therapeutically useful gene” is a gene encoding a nucleic acid or polypeptide that, when expressed in a cell, for example, a target host cell, can provide a therapeutic effect.

A molecule that specifically binds to a second molecule (e.g., to a particular receptor on a cell) is a molecule that the second molecule, but does not substantially bind other molecules in a sample, e.g., a biological sample, which naturally contains the second molecule.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In addition, the materials, methods, and examples are illustrative only and not intended to be limiting.

Other features and advantages of the invention will be apparent from the detailed description, and from the claims.

### DESCRIPTION OF DRAWINGS

FIG. 1 is a bar graph illustrating the results of an experiment in which NIH 3T3 cells and A375 human melanoma cells were transduced by RGD₁₃ viruses.

FIG. 2 is a bar graph showing the results of experiments testing the ability of RGD₂₁ viruses to transduce NIH 3T3 cells and A375 human melanoma cells.

FIGS. 3A-3B are bar graphs illustrating transduction experiments testing the requirement of the RGD sequence for transduction of human cells. (A) Transduction of NIH 3T3 infected with an RGD₂₁ or RGE₂₁ virus, and (B) Transduction of A375 human melanoma cells infected with an RGD₂₁ or RGE₂₁ virus.

FIGS. 4A-4B are bar graphs showing the results of experiments testing the effect of pretreatment with antibodies to integrin receptors on transduction of human cells by RGD viruses (A) NIH 3T3 cells; (B) A375 human melanoma cells.

FIG. 5 is a bar graph showing the results of experiments testing the ability of GRP viruses to transduce human cells.

FIGS. 6A-6C are bar graphs showing the results of experiments examining the requirement of the GRP receptor for transduction of human cells by GRP viruses. (A) Antibodies to GRP block transduction of human cells by GRP viruses. (B) Requirement of the GRP receptor for transduction of human 293 cells. (C) Requirement of the GRP receptor for transduction of mouse cells by GRP-2, GRP-3 and GRP-5 viruses.

FIGS. 7A-7B are bar graphs showing the results of experiments testing the ability of HRG viruses to transduce NIH 3T3 cells and MDA-MB-453 breast carcinoma cells. (A) Transduction of NIH 3T3 cells by HGR viruses. (B) Transduction by HRG-1 or HRG-8 virus after pretreatment of NIH 3T3 and MDA-MB-453 breast carcinoma cells with antibodies to HER-3 and HER-4 receptors.

FIG. 8 is a representation of the nucleic acid sequence of MoMLV envelope protein (SEQ ID NO:4).

### DETAILED DESCRIPTION

The invention provides a strategy for altering the host range of ecotropic retrovirus vectors using a recombinant envelope protein that contains a heterologous short peptide ligand (chimeric envelope proteins). Viruses expressing such chimeric envelope proteins (pseudotyped virus) can transduce human cells without removal of the N-terminal region of the naturally occurring envelope protein or co-expression of wild-type envelope protein. Furthermore, it is not necessary to delete portions of the wild-type envelope protein sequence to obtain a chimeric envelope protein that, when present in a pseudotyped virus, can alter host specificity and infect with reasonable efficiency. Depending on the site in the envelope protein of insertion of the heterologous short peptide ligand, the pseudotyped virus containing the resulting chimeric envelope protein can transduce only target host cells. Target host cells can be any eukaryotic cell type expressing a sequence on the cell surface that can bind to the heterologous short peptide ligand. In general, a target host cell is a mammalian cell, e.g., a human cell. In one embodiment, a heterologous short peptide ligand is inserted into an extracellular portion of an MLV envelope protein. For example, the heterologous short peptide ligand can be inserted into a conserved region of the envelope protein, or into a variable region.

Heterologous Short Peptide Ligands

Heterologous short peptide ligands for use in the invention can be those already identified in the art. Many peptide sequences that bind to cell surface proteins have been identified. Some such sequences are so-called “designer” peptides whose affinity for receptors surpasses that of wild-type peptide sequences. One example of such a designer peptide is the heregulin peptide described in Table 2 and Example 8. Additional examples of cell surface proteins/receptors that bind to ligands are include flt-3 receptor/flt3 ligand (FL), transferrin receptor/transferrin, erythropoietin receptor/erythropoietin (EPO) peptides (e.g., the consensus sequence IEGPTLRQWLAARA; SEQ ID NO:1; Cwirla, et al., 1997, Science 276:1696-1699), CD34/variable sequence of a binding antibody; c-kit/stem cell factor (binding region peptide); human melanoma-associated chondroitin sulfate proteoglycan (MCSP)/anti-MCSP antibody (used for the detection of antibodies); MHC class I/Semiliki Forest Virus binding sequence; MHC class II low density lipoprotein receptor/variable sequence of antibody; mucins (surface glycoproteins overexpressed in numerous cancers)/binding peptide sequence (APDTP; SEQ ID NO:2); IL-2 receptor/IL-2; surface glycoprotein high-molecular-weight melanoma-associated antigen (HMW-MAA)/binding region from variable sequence of antibody.

Heterologous short peptide ligands suitable for use in the invention can also be identified using methods known in the art. Such methods include screening phage selected for binding to the extracellular domain of a cell surface protein (i.e., a cell surface protein expressed on a host target cell). Nucleic acid sequences coding for such peptides are then cloned into wild-type envelope protein to produce chimeric envelope proteins. In another method using phage library, targeting to various organs can be achieved by injecting a phage display library into animals and identifying the peptides localized in each organ. This method has been successfully used to identify short peptides targeted to, e.g., kidney cells (CLPVASC, SEQ ID NO:3; and CGAREMC, SEQ ID NO:5) and to brain cells (CLSSRLDAC, SEQ ID NO:6; WRCVLREGPAGGCAWFNRHRL; SEQ ID NO:7) (Pasqualini et al., 1996, Nature 380:364-366). Similarly, recombinant peptide libraries can also be screened for peptides that specifically bind to a protein that is expressed on a target host cell (Pasqualini supra; Wrighton et al., 1996, Science 273:458-464; Cwirla et al., 1997, Science 276:1696-1699; Arap et al., 1998, Science 279:377-380).

Chimeric Envelope Proteins and Libraries

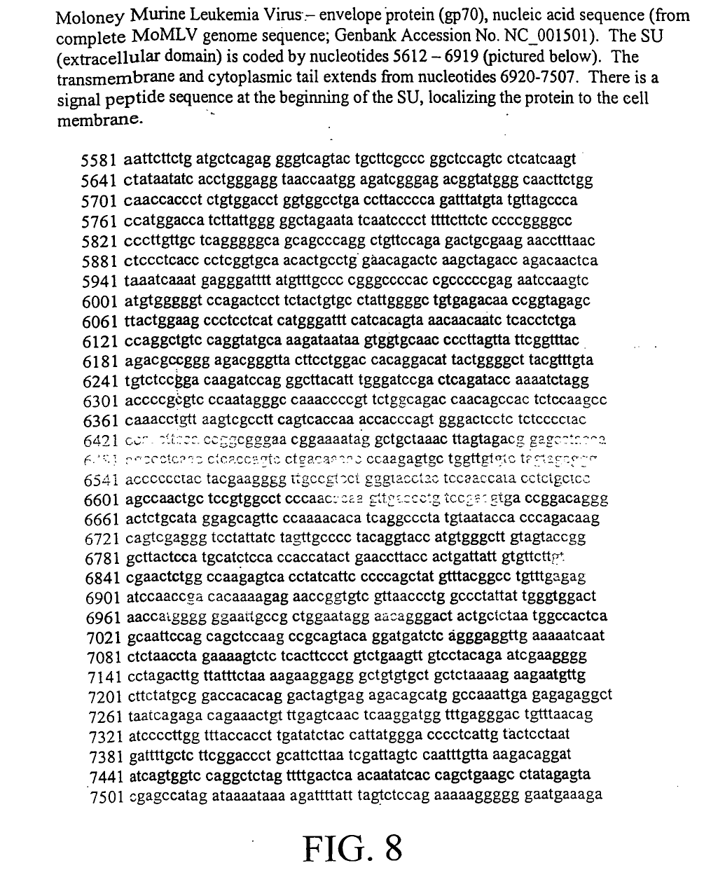

Envelope proteins are known in the art. In particular, the ecotropic murine leukemia virus protein has been extensively studied. The sequence of the MoMLV envelope protein (gp70) is shown in FIG. 8. The sequence coding for the extracellular domain (SU) region of the envelope protein extends from nucleotides 5612-6919. The transmembrane region and cytoplasmic tail extend from nucleotides 6920-7507. There is a signal peptide sequence at the beginning of the SU, that localizes the protein to the cell membrane. Clones containing MoMLV envelope protein are commercially available (e.g., Stratagene, La Jolla, Calif.). Heterologous short peptide ligands are inserted in the extracellular domain of the envelope protein. In general, chimeric envelope proteins containing insertions near the N-terminus and in the proline-rich region (PRR region) of the envelope protein are less effective for altering viral tropism than insertions at other positions within the protein. Examples of specific insertion locations that are effective are described herein, and in detail in the Examples.

Transduction efficiency also depends on the presentation of the ligand within the envelope. In some embodiments of the invention, cysteine residues flank the inserted heterologous short peptide ligand. Such residues are expected to form a disulfide bond that facilitates ligand presentation. Cysteines that flank the heterologous short peptide ligand can be immediately adjacent to the short peptide sequence. In some embodiments of the invention, such sequences are 2, 3, 4, 5, or about 10, 20, 30, 50, or 100 amino acid residues from the ends of the heterologous short peptide ligand. The cysteines can be added to the envelope protein that is being engineered to contain the heterologous short peptide ligand, or the heterologous short peptide ligand can be positioned so that one or two cysteines that naturally occur in the wild-type protein are flanking cysteines.

The invention includes the generation and screening of chimeric envelope protein libraries. In one method of generating such libraries, a cloned envelope protein (e.g., a cloned MoMLV envelope protein) or a portion of an envelope protein, generally the sequence coding for the extracellular domain of the envelope protein, is cut with restriction enzyme. Typically, the restriction enzyme(s) are four-base cutters and the reaction is carried out in the presence of ethidium bromide. The presence of ethidium bromide limits the number of times a plasmid will be cut by the restriction enzyme, typically to one cleavage per plasmid, thus resulting in linearized plasmid. The ends of the plasmid are treated to produce blunt ends. A blunt-ended nucleic acid sequence encoding the heterologous short peptide ligand of interest is prepared and ligated into the linearized plasmid preparation. Different restriction enzymes can be used to increase the number of sites into which sequences coding for the heterologous short peptide ligand can be inserted. The plasmids can then be transfected into bacteria. Plasmids are examined for heterologous short peptide ligand sequence and the location of the heterologous short peptide ligand within the envelope sequence using methods known in the art, e.g., PCR and Southern blot analysis. If a portion of the envelope sequence was used for construction of a sequence containing the inserts of heterologous short peptide ligand, then the portion of the envelope sequence containing the heterologous short peptide ligand is cloned into a plasmid containing envelope sequence to generate a sequence coding for a complete envelope protein containing a heterologous short peptide ligand (i.e., a chimeric envelope protein).

Pseudotyped Viruses

To produce pseudotyped viruses containing a specific chimeric envelope protein, a plasmid that contains a sequence that codes for the chimeric envelope protein is co-transfected into a packaging cell with a packaging construct, e.g., the packaging cell line, Anjou 65 (Pear et al., 1993, Proc. Natl. Acad. Sci. USA 90:8392-8396) and the packaging construct LGRNL (Yee et al., 1994, Methods Cell Biol. 43:99-112). The resulting cell is maintained under conditions such that virus is produced. The resulting pseudotyped virus can then be tested for its ability to transduce a natural host cell (e.g., a murine cell when the pseudotyped virus is derived from an ecotropic virus) and a target host cell (e.g., a human cell). A virus may be able to transduce target host cells from more than one species, depending on the ability of the heterologous short peptide ligand to bind to the corresponding receptors expressed on cells from various species. A pseudotyped virus may also transduce more than one cell type, e.g., those cell types that express the targeted receptor.

Cells can be tested for transduction using methods known in the art. For example, Southern blotting can be used to test for insertion of retroviral sequence into a host cell genome. The pseudotyped virus may include a selectable gene, e.g., a gene that confers drug resistance such as neo. In this case, an infected host cell is incubated in the presence of the drug. Cells that have been successfully transduced survive in the presence of the drug. Pseudotyped virus can also be tested for the efficiency of transduction. In general, pseudotyped viruses with the greatest efficiency of transduction of host cells are preferred, e.g., for delivery of a gene to a cell as in gene therapy.

In some cases, it is desirable to introduce an additional gene, e.g., a therapeutically useful gene, into the pseudotyped virus and/or into the packaging cell. Such a gene can be either on the packaging construct or on a separate plasmid. Therapeutically useful genes include those that replace or supplement the product of a defective gene in the target host cell. Examples of such genes include the globin genes delivered to bone marrow progenitor cells to treat sickle cell anemia or a thalassemia, and factor VIII or factor IX genes delivered to blood progenitor cells to treat hemophilia. Also included are genes that encode proteins, antisense transcripts, or ribozymes that can be delivered to cells that express CD4 and can be used to treat HIV, genes that encode therapeutic antibodies, growth factors, or cytokines to be expressed by host target cells. Therapeutically useful genes also include genes that can be used for cancer therapy such as genes that code for proteins that destroy the target host cell (either directly or after treatment of such cells with a drug) and genes that code for antisense transcripts or ribozymes that interfere with target host cell function.

Gene Delivery

Pseudotyped viruses as described herein are useful as vectors for delivery of genes to cells, e.g., for ex vivo or in vivo gene therapy. In addition to the advantages conferred by using a retrovirus (e.g., integration of the transferred gene(s)) an advantage of the pseudotyped viruses is that they can be designed to transduce specific cell types. For example, as discussed supra, some cancer cells overexpress specific cell surface proteins. Such proteins can be used as the target receptors for a heterologous short peptide ligand in the chimeric envelope protein, thus conferring specificity on a pseudotped virus that expresses the chimeric envelope protein. A further advantage of using an envelope protein from a murine ecotropic virus for making the chimeric envelope proteins, is that the naturally occurring envelope protein will target only murine cells. Thus, the pseudotyped virus, if used to infect a non-murine cell such as a human cell, will transduce only those cells expressing the receptor for the heterologous short peptide ligand. Such pseudotyped viruses whose tropism has been altered can also be selected for the inability to transduce a murine cell and the ability to transduce a cell expressing a target receptor. As described herein, it is also possible to make and identify, depending on the location of the heterologous short peptide ligand within the chimeric envelope protein, pseudotyped virus that can only transduce a target host cell, i.e., cannot transduce a murine cell.

Pseudotyped viruses made using the methods described herein can be used to introduce a gene into an animal or into cells of an animal that are cultured in vitro then reintroduced into the animal (ex vivo gene therapy). In addition, a pseudotyped virus that contains a therapeutically useful gene can be introduced into an animal model for a disease such as cancer. Therapeutically useful genes are discussed supra and include genes that code for a protein that is defective in the animal or for a gene that provides a novel property to a cell, for example, drug sensitivity to a tumor cell. The pseudotyped virus may be introduced using any method known in the art. For example, the pseudotyped virus can be introduced locally (for example, near a tumor) or systemically. In the latter case, it may be desirable to immunosuppress the animal using methods known in the art to minimize the immune response to the pseudotyped virus.

The use of retroviral vectors is known in the art and the pseudotyped viruses described herein provide advantages over the presently used vectors. In particular, the target cell specificity and the limited ability of the pseudotyped vectors to replicate in target host cells are an improvement over those systems in which the viral vector infects cells other than those where gene delivery is desired and in which viral replication may interfere with the cellular metabolism.

Gene therapy vectors can be delivered to a subject by, for example, intravenous injection, local administration (see U.S. Pat. No. 5,328,470), or by stereotactic injection (see e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is embedded. Alternatively, where the complete gene delivery vector (e.g., a pseudotyped virus) can be produced intact from recombinant cells, the pharmaceutical preparation can include one or more cells that produce the gene delivery system.

The pharmaceutical compositions can be included in a container, pack, or dispenser together with instructions for administration.

Viruses

In general, the invention uses envelope proteins derived from ecotropic retroviruses such as the Moloney murine leukemia virus (MoMLV). Also useful in the invention are viruses that express a glycoprotein envelope. Such viruses include the murine leukemia virus family (MLV) (e.g., amphotropic, ecotropic, and xenotropic viruses). Amphotropic viruses can typically infect human cells, whereas ecotropic viruses can infect only host cells of the species in which they originated. Thus, murine ecotropic viruses cannot naturally infect human cells. Host targeting (tropism) of viruses other than retroviruses can also be modified using envelope proteins. Examples of such viruses include adenovirus (by inserting a heterologous short peptide ligand into the fiber of the surface protein of adenovirus) and vesicular stomatitis virus (VSV-G), which is an attractive candidate as the pseudotyped virus can be concentrated by high speed centrifugation without significant loss of titre. Cells (e.g., human) can be targeted using pseudotyped viruses derived from many different viruses, including those that that enter the cell through an endocytic process (e.g., Moloney MLV, as described herein), or by a virus that fuses at the cell surface (as with amphotropic MLV). Additional examples of viruses whose targeting can be modified using the methods described herein include gibbon ape leukemia virus, influenza virus (chimeric hemagglutinin), spleen necrosis virus, reticuloendotheliosis virus strain A (REV-A), herpes virus (HSV-1), human immunodeficiency virus (HIV; Naldini et al., 1996, Science 272: 263-267), and various species of hepatitis virus.

In general, a library of chimeric envelope proteins containing heterologous short peptide ligands that are useful for altering host range of a virus are made as described herein. The chimeric envelope proteins can first be screened for their ability to bind to the receptor to which the heterologous short peptide ligand binds when it is not inserted into an envelope protein. The chimeric envelope proteins are incorporated into a virus (thus making a pseudotyped virus) and tested for their ability to specifically transduce a target host cell.

Uses of Pseudotyped Viruses

It is demonstrated herein that chimeric envelope proteins enable transduction of human cells by a pseudotyped virus derived from an MLV. In addition, transduction of human cells with pseudotyped MLV does not occur with heterologous short peptide ligand insertions (e.g., RGD peptide ligands) in the PRR (proline-rich region) or C-terminal region of the envelope, although pseudotyped viruses containing such insertions can transduce mouse cells. Some viruses bearing insertions (e.g., of RGD peptide ligands) at the N-terminus or VRA region (RGD₁₃-4,5,8 and RGD₂₁-2) transduce human but not mouse cells. Thus, the position of the inserted ligand can dictate tropism.

Transduction efficiencies differ between different RGD pseudotyped viruses, indicating that the precise location of the ligand within envelope is important. In one aspect, the invention includes methods for optimizing the location of heterologous short peptide ligands (e.g., RGD peptide ligands) within the envelope protein. In general, RGD₁₃ and RGD₂₁ ligands transduce NIH 3T3 cells with comparable efficiencies. Thus, the envelope protein can accommodate ligands of different sizes and remain effective for transduction. Longer ligands can be more disruptive to the structure of the envelope protein, but may also have increased affinity for the target receptor. Such ligands can include repeats of the heterologous short peptide ligand sequence (for example, 2 copies, three copies, 4 copies, five copies, or up to ten copies).

The methods described herein for altering the tropism of a retrovirus can be used to selectively target and destroy human cancer cells. For example, many cancer cells overexpress specific cell surface receptors. As discussed below, Moloney murine leukemia virus (MLV) envelope proteins bearing heterologous short peptide ligands for gastrin releasing protein (GRP) and human epidermal growth factor receptors (HRG) were generated. More than twenty MLV chimeric envelope proteins that contain the GRP or a modified HRG peptide ligand were inserted at various locations within envelope. Pseudotyped viruses containing these chimeric envelope proteins selectively transduce human cancer cell lines that overexpress the cognate receptor. For both GRP and HRG viruses, some insertions within the N-terminal region or VRA (a variable region) of the envelope protein interfere with transduction of mouse cells. Several of these GRP viruses transduce cells expressing the GRP receptor indicating that tropism is altered. Thus, for production of selective targeting retroviral vectors, the N-terminal region and VRA can be the optimal locations for ligand insertion. Transduction by viruses containing the larger HRG ligand is, in general, decreased relative to their GRP counterparts and several HRG viruses are unable to transduce mouse or human cells. However, the HRG ligands used in these experiments are approximately twice as long as the GRP ligands. This further demonstrates that short ligands are generally more efficient for use in the methods of the invention and are an improvement over those generally used previously.

The new methods include using a pseudotyped virus containing a chimeric envelope protein to deliver a therapeutically useful gene to a cell. This was demonstrated by showing that pseudotyped virus targeting the GRP receptor can deliver the thymidine kinase (TK) gene to human melanoma and breast cancer cells, which makes these transgenic cells susceptible to the antiviral agent, ganciclovir. Furthermore, the transduced cells were killed by the subsequent addition of ganciclovir, demonstrating that heterologous short peptide ligands inserted at appropriate locations in an ecotropic envelope protein (e.g., MoMLV envelope protein) can selectively target a retrovirus to a human cancer cell and deliver a therapeutically useful gene. These experiments also demonstrate the utility of the method and constructs to selectively target cancer cells overexpressing GRP or HRG receptors and deliver a therapeutically useful gene. The method can also be used, e.g., to introduce a gene or other nucleic acid sequence into any cell type that expresses a receptor that can be targeted as described herein. This includes introducing a gene or other nucleic acid into a cell in culture or in an animal (e.g., a non-human mammal such as a mouse, rat, sheep, cow, or goat). For example, in a mixed culture of cells, the method can be used to deliver a gene to a single cell type in the culture, e.g., to provide a marker for the cell type or to introduce a drug-resistance gene to that cell type.

A pseudotyped virus containing a chimeric envelope protein can be generally useful in gene therapy methods for animals and humans. Gene therapy strategies have been proposed for many human diseases, including rare heritable genetic defects, of which there are more than 4000, and many common diseases including cancer, AIDS, hypertension, and diabetes (Anderson, 1992, Science 256: 808-813; Friedmann, 1992, Nature Genet. 2:93-98; Russell, 1993, Cancer J. 6:21-25). The invention therefore has an important application in many areas of human medicine.

### EXAMPLES

### Example 1

### Cell Lines

In experiments described herein, Anjou 65 (Pasqualini and Ruoslahti, 1996, Nature 380:364-366), NIH 3T3, XC cells (Wrighton et al., 1996, Science 273:458-464), A375 human melanoma, HT 1080 human fibrosarcoma, and MDCK canine kidney cells were each cultured separately as monolayers in Dulbecco's modified Eagle medium (DMEM; Gibco BRL) supplemented with 10% fetal bovine serum (Hyclone), 2 mM glutamine, and 5 mM HEPES. All cell lines, except for Anjou 65, were obtained from the American Type Culture Collection (ATCC) and maintained at 37° C. in a 5% CO₂ atmosphere.

### Example 2

### Construction of Short Peptide RGD Ligand Viruses

To test for the ability of heterologous short peptide ligands to redirect the host range of a virus, more than 40 chimeric envelope proteins containing in-frame insertions of either a 13 or 21 amino acid RGD peptide (RGD₁₃ or RGD₂₁, respectively; Table 1) were examined. The sequences of the RGD₁₃, RGD₂₁, and RGE₂₁ ligands are shown in Table 1. For the chimeric envelope proteins RGD₁₃ 1-26, RGD₂₁ 1-16 and RGE₂₁ 1-5, the position of ligand insertion, number of inserts, and any additional modifications are indicated in Table 1.

The heterologous short peptide ligands were introduced into envelope protein to form chimeric envelope proteins. To construct the chimeric envelope proteins, the extracellular domain (gp70) of ecotropic MLV envelope gene was linearized at random locations by partial digestion with blunt-end restriction endonucleases in the presence of 50 to 400 ng/ml ethidium bromide. The 13 amino acid RGD sequence (CAAAGRGDSPTRC; RGD₁₃; SEQ ID NO:8) was derived by annealing two oligonucleotides, RGD₁₃-A (TGCGCGGCCGCTGGCCGTGGCG-ATTCTCCCACGCGTTGT; SEQ ID NO:9) and RGD₁₃-B (ACAACGCGTGGGAGAATCGCC-ACGGCCAGCGGCCGCGCA; SEQ ID NO:10). The annealed sequence was ligated into the linearized envelope plasmid and subclones screened for insert position and orientation using standard techniques. The resultant chimeric envelope proteins were cloned into the envelope expression vector, pCEE (MacKrell et al., 1996, J. Virol. 70:1768-1774). The RGD₁₃-3 chimeric envelope proteins were constructed by insertion of a Nae I linker at the C-terminus of the signal sequence of wild type envelope and the annealed RGD oligonucleotides were cloned into the Nae I site. Chimeric envelope proteins with the 21 amino acid RGD sequence CAAAQGATFALRGDNPQGTRC; RGD₂₁; SEQ ID NO:11) were constructed by restriction endonuclease digestion of RGD₁₃ envelopes with Not I and Mlu I and insertion of the RGD₂₁ annealed oligonucleotidesRGD21-A (GGCCGCTCAAGGCGCAACGTTCGCGCTC-AGAGGCGATAATCCACAGGGGA; SEQ ID NO:12) and RGD21-B (CGCGTCCCCTGT-GGATTATCGCCTCTGAGCGCGAACGTTGCGCCTTGAGC; SEQ ID NO:13). The RGD₂₁ envelope proteins were cloned into an expression plasmid that contained a Zeocin™ selection marker (Invitrogen, Carlsbad, Calif.). RGE₂₁ was constructed using methods analogous to those used for RGD₂₁. Chimeric envelope proteins expressing two RGD sequences, RGD₂₁-15 and RGD₂₁-16, were constructed by removal of the Bst EII/Cla I fragment of RGD₂₁-1, and insertion of the Bst Eli/Cla I region from RGD₂₁-4 and RGD₂₁-9, respectively.

TABLE 1
Description of RGD viruses.

| | | | |
|---|---|---|---|
| | Position | | |
| | of Ligand | | |
| | Insertion | | |
| | (A.A. | Deletion of | |
| ENV # | Location) | #of Inserts | Nucleotides in Env. |

| | |
|---|---|
| RGD₁₃[CAAA-GRGDSP-TRC] | (SEQ ID NO:8) |

| | | | |
|---|---|---|---|
| 1 | 1 | 1X | |
| 2 | 1 | 2X | |
| 3 | 1 | 4X | |
| 4 | 38 | 1X | |
| 5 | 38 | 3X | |
| 6 | 38 | 1X | 5990-6082 |
| 7 | 68 | 1X | |
| 8 | 68 | 2X | |
| 9 | 68 | 1X | 6082-6191 |
| 10 | 120 | 1X | |
| 11 | 120 | 2X | 6238-6281 |
| 12 | 120 | 3X | |
| 13 | 185 | 1X | |
| 14 | 230 | 1X | |
| 15 | 230 | 2X | |
| 16 | 235 | 1X | |
| 17 | 235 | 4X | |
| 18 | 310 | 1X | |
| 19 | 310 | 2X | |
| 20 | 321 | 1X | |
| 21 | 321 | 2X | |
| 22 | 382 | 1X | |
| 23 | 382 | 2X | |
| 24 | 382 | 3X | |
| 25 | 388 | 1X | |
| 26 | 388 | 2X | |

| | |
|---|---|
| RGD₂₁[CAAA QGATFALRGDNPQG-TRC] | (SEQ ID NO:11) |

| | | | |
|---|---|---|---|
| 1 | 1 | 1X | |
| 2 | 38 | 1X | |
| 3 | 38 | 1X | 5990-6082 |
| 4 | 68 | 1X | |
| 5 | 68 | 1X | 6082-6191 |
| 6 | 120 | 1X | |
| R | 120 | 1X | 6238-6281 |
| 8 | 185 | 1X | |
| 9 | 230 | 1X | |
| 10 | 235 | 1X | |
| 11 | 310 | 1X | |
| 12 | 321 | 1x | |
| 13 | 382 | 1X | |
| 14 | 388 | 1X | |
| 15 | 1,68 | 1X,1X | |
| 16 | 1,230 | 1X,1x | |

| | |
|---|---|
| RGE₂₁[CAAA-QGATFALRGENPQG-TRC] | (SEQ ID NO:25) |

| | | | |
|---|---|---|---|
| 1 | 1 | 1X | |
| 2 | 38 | 1X | 5990-6082 |
| 3 | 68 | 1X | |
| 4 | 68 | 1X | 6082-1916 |
| 5 | 230 | 1X | |

The core of the RGD₁₃ ligand is a six amino acid peptide, GRGDSP (SEQ ID NO:14), which represents an RGD consensus sequence. The core of the RGD₂₁ ligand is a 14 amino acid sequence, QGATFALRGDNPQG (SEQ ID NO:15), derived from the mouse laminin protein (Aumailley et al., 1990, FEBS Lett. 262:82-86). Both the RGD₁₃ and RGD₂₁ peptides were flanked by cysteine residues to constrain the sequence within a loop (Aumailley et al., 1990, supra; Yamada et al., 1993, J. Biol. Chem. 268:10588-10592; Hart et al., 1994, J. Biol. Chem. 269:12468-12474; Pierschbacher and Ruoslahti, 1987, J. Biol. Chem. 262:17294-17298).

In some cases, chimeric envelope proteins with multiple ligands in tandem were also generated. Several of the chimeric envelope proteins had deletions of envelope sequences, in addition to ligand insertions, as a result of multiple restriction enzyme cleavages. In total, 26 chimeric envelope proteins containing the RGD₁₃ ligand, 16 chimeric envelope proteins containing the RGD₂₁ ligand, and five chimeric envelope proteins containing an RGE₂₁ ligand, a control non-binding peptide (Aumailley et al., 1990, supra; Hart et al., 1994, supra; Solowska et al., 1989, J. Cell Biol. 109:853-861; Greenspoon et al., 1993, Biochemistry 32:1001-1008), were constructed.

The information provided in this Example provides guidance for construction of chimeric envelope proteins containing heterologous short peptide ligands.

### Example 3

### Transduction of Cells with Viruses Containing Chimeric Envelope Proteins

Pseudotyped viruses were generated that express the chimeric envelope proteins as were control viruses that expressed wild-type ecotropic envelope protein and that expressed the envelope protein from an amphotropic virus. None of the pseudotyped viruses contained a wild type envelope gene. This feature provides an advantage for altering viral tropism since all of the envelope genes in the pseudotyped virus will contain the heterologous short peptide ligand, thus providing more sites for binding to the target host cell.

Plasmids used to express control ecotropic virus, ECO (wild type), were generated by expressing the wild-type ecotropic envelope gene encoded by the plasmid pCEE. Another control was an amphotropic virus, AMPH, which contains an amphotrophic viral envelope protein. This virus was generated by expressing the amphotropic envelope, encoded by the expression vector pCAA The pCAA expression vector was generated by removing the amphotropic envelope gene from a full-length infectious clone (Ott et al., 1990, J. Virol. 64:757-766) and engineering it into the expression vector.

A packaging construct for use in the experiments, LAPNL, was generated by removal of the VSV-G envelope from LGRNL (Yee et al., 1994, Methods Cell Biol. 43:99-112) and insertion of the secreted alkaline phosphatase gene (SEAP) into LGRNL, producing the packaging construct LAPNL. Transfection with this packaging construct was measured by assaying for the secreted alkaline phosphatase. The SEAP assay was performed as described by Tropix, Inc. and measured in a luminometer (Moonlight 2010, Analytical Luminescence Laboratory).

Pseudotyped virus containing chimeric envelope proteins was generated using a human 293T cell-based packaging cell line, Anjou 65 (Pear et al., 1993, Proc. Natl. Acad. Sci. USA 90:8392-8396). The pseudotyped virus producer cell lines were generated by cotransfection of Anjou 65 cells with LAPNL and a plasmid expressing a chimeric envelope protein using Dotap (Boehringer) followed by selection in Zeocin™ (200 μg/ml) for two weeks. RGD₁₃ required cotranfection with a Zeocin expression plasmid (Invitrogen, Carlsbad, Calif.).

Pseudotyped virus was harvested from viral producer cell lines. Virion associated reverse transcriptase (RT) activity was performed as previously described to measure RT activity of harvested viral supernatant. RT/PCR was performed by first generating cDNA from 5 μl of harvested pseudotyped virus using the protocol for Superscript II (Gibco BRL) and oligo-dT with 0.1% NP40. Oligonucleotides to the neomycin gene in the LAPNL packaging vector were used to generate the PCR product from the cDNA. These oligonucleotides were labeled N1 (TTTTGTCAAGACCGACCTGTCC; SEQ ID NO:16) and N2 (CGGGAGCGGCGATACCGTAAAG; SEQ ID NO:17). Target cells were infected as described in Kasahara et al. (1994, Science 266:1373-1376) and Cosset et al. (1995, J. Virol. 69:6314-6322). Briefly, 24 hours before infection, NIH 3T3 and A375 human melanoma cells were seeded on 60 mm plates at 2×10⁵ cells/plate. Infected cells were seeded onto 150 mm plates and selected for two weeks with 1.0 mg/ml of G418. Colonies were fixed and stained with Giemsa as described in Russell et al. (1993, Nucleic Acids Res. 21:1081-1085). Human fibrosarcoma HT 1080 cells and canine kidney cells MDCK were also infected, examined, and selected in 600 μg/ml of G418. Transduction efficiency was determined by SEAP measurements and by counting colonies using a BioRad digital camera and scanner.

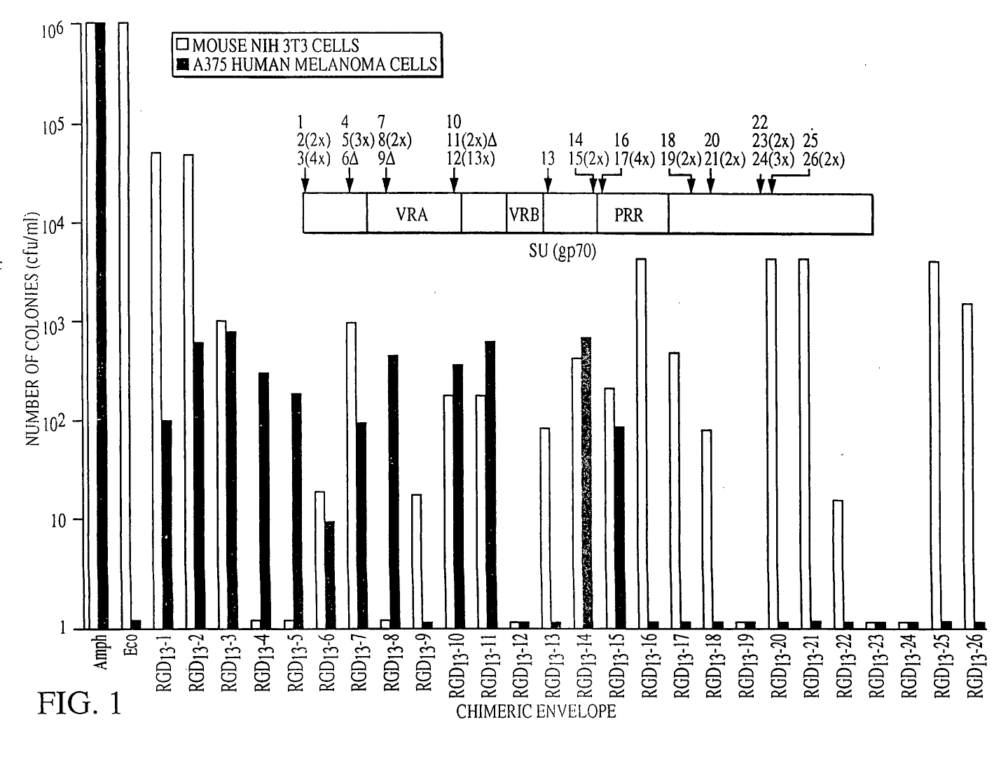

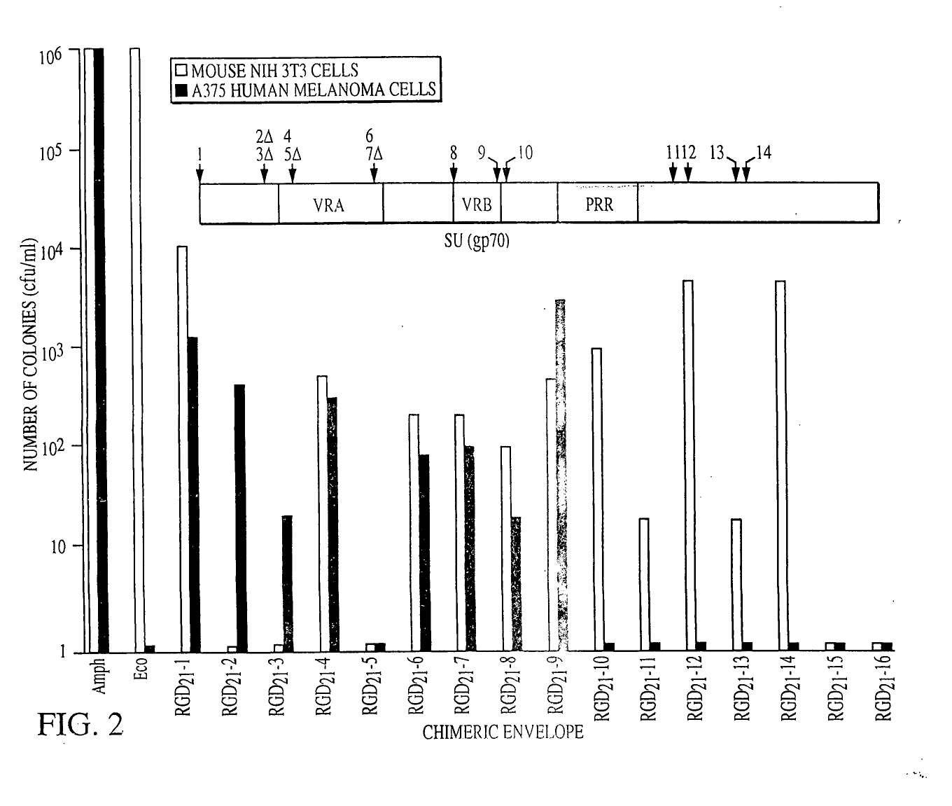

Immunoblotting of purified virions indicated that, in all cases tested, the chimeric envelope proteins were incorporated into the virion and correctly processed. The viruses expressing the chimeric envelope protein with short RGD peptide ligand (RGD viruses) were initially tested for their ability to transduce mouse NIH 3T3 cells. Data from the mouse cell transduction experiments are shown in FIGS. 1 and 2. These data show that many of the RGD viruses retained their ability to transduce mouse cells but those bearing insertions within the N-terminus (RGD₁₃-4,5; RGD₂₁-2,3), VRA (RGD₁₃-8,12; RGD₂₁-5) and C-terminal region (RGD₁₃-19,23,34; RGD₂₁-15,16) did not. Several of these latter RGD viruses also failed to transduce human cells (RGD₁₃-12,19,23,24; RGD₂₁-5,15,16), whereas for others (RGD₁₃-4,5,8; RGD₂₁-2,3) the defect was mouse cell specific. In addition, most RGD₂₁ viruses transduced NIH 3T3 cells with comparable efficiencies to the equivalent RGD₁₃ viruses, and none of the RGD₂₁ viruses transduced NIH 3T3 cells with greater efficiency than the equivalent RGD₁₃ virus.

Thus, chimeric envelope protein containing a heterologous short peptide ligand, when expressed in a packaging system can effectively infect a cell from an organism other than the natural host of the parent virus, thus the host range of the virus can be altered by creating viruses with heterologous short peptide ligands in their envelope protein.

### Example 4

### Transduction of Cells Expressing Integrin Receptors

To further assess the ability of RGD viruses to infect non-mouse cells, the viruses were tested for their ability to transduce A375 human melanoma cells. A375 cells have been used to study integrin receptor binding (Gehlsen et al., 1992, Clin. Exp. Metastasis 10:111-120; Pfaff et al., 1993, Exp Cell Res. 206:167-176; Allman et al., 2000, Eur. J. Cancer 36:410-422). As expected, viruses containing unmodified MLV envelope failed to transduce this human cell line. Significantly, however, many of the RGD viruses were able to transduce A375 human melanoma cells (FIGS. 1 and 2). Transduction occurred when the RGD peptide was inserted at the N-terminus (RGD₁₃ 1-3; RGD₂₁-1), within the N-terminal region (RGD₁₃ 4-6; RGD₂₁-2,3), within the VRA region (RGD₁₃-7,8,10,11; RGD₂₁-4,6,7), and upstream of the PRR (RGD₁₃-14,15; RGD₂₁-8,9).

RGD viruses with insertions in the PRR (proline-rich region) and C-terminal region failed to transduce human cells. Thus, in constructing chimeric envelope proteins, the PRR is generally not a preferred site for insertion of a heterologous short peptide ligand. Several of the RGD viruses that transduced human cells, failed to transduce NIH 3T3 cells (RGD₁₃-4,5,8 and RGD₂₁-2), indicating that viral tropism can be eliminated for the natural host and altered to target a different host.

In all cases tested, RGD viruses that transduced A375 human melanoma cells also transduced other human and non-human cell lines that contained integrin receptors. This shows that the host range for a virus can be greatly changed and expanded by introducing a chimeric envelope protein containing a heterologous short peptide ligand. Furthermore, these viruses can be targeted to infect a specific host cell.

### Example 5

### Specificity of Transduction by Virus Containing Chimeric Envelope

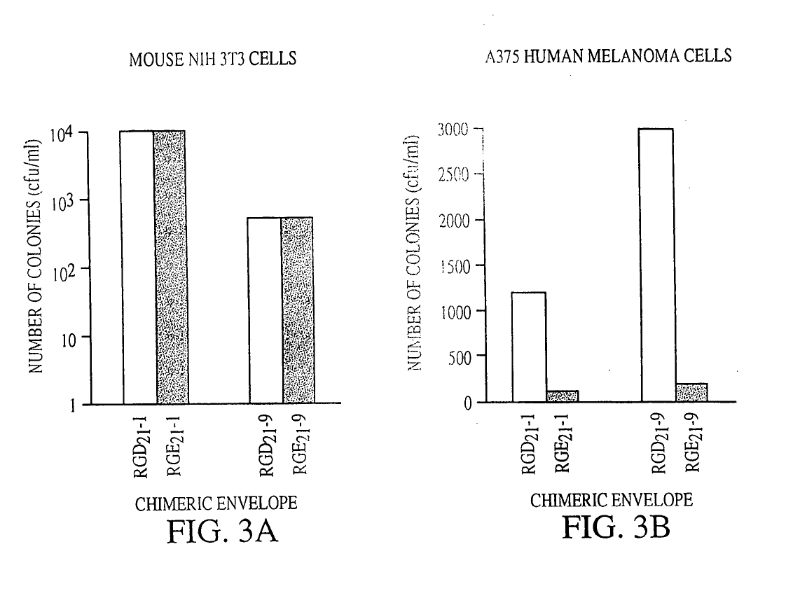

To examine the basis and specificity of human cell transduction, two experimental approaches were undertaken. In the first approach, the RGD₂₁ ligand was replaced with the corresponding RGE₂₁ sequence. Pseudotyped virus expressing an RGE₂₁ chimeric envelope derivative transduced NIH 3T3 host cells with efficiencies comparable to the equivalent RGD₂, derivative. However, transduction of A375 human melanoma cells was significantly reduced (FIG. 3).

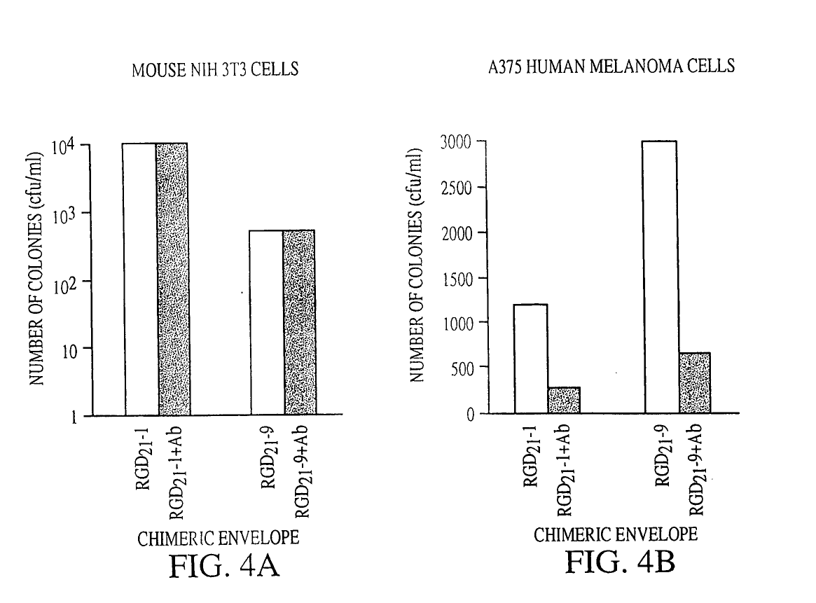

In a second approach, the effect on transduction with RGD viruses was examined in the presence of antibodies that bind integrin receptors. In these experiments, NIH 3T3 and A375 human melanoma cells were pretreated with integrin receptor antibodies, and transduction was performed with two of the RGD₂₁ viruses. Briefly NIH 3T3 and A375 human melanoma cells were pretreated with polyclonal antibodies to β₁, β₃, and αᵥ integrin receptors (Santa Cruz Biotechnology). For pretreatment, the three antibodies were diluted 1:100 in DMEM medium and incubated with the cells for four hours. Cells were then incubated with pseudotyped virus (RGD₂₁-1, RGD₂₁-4, or RGD₂₁-9) for six hours. The infected cells were then analyzed for transduction as described above (see FIG. 1). It was observed that transduction of human but not mouse cells was substantially reduced (FIG. 4).

### Example 6

### Chimeric Envelope Proteins Containing GRP Heterologous Short Peptide Ligands

To test the applicability of the invention to heterologous short peptide ligands in addition to integrin ligands, heterologous short peptide ligands from bombesin (GRP) and heregulin (HRG) were identified and cloned into MLV ecotropic envelope using methods known in the art.

The sequence of the GRP and HRG ligands are shown in Table 2. A 21 amino acid GRP sequence, containing 14 residues of the bombesin protein, was inserted at various locations within the MLV ecotropic envelope to generate 14 GRP chimeric envelope proteins (Table 2). For the chimeric envelope proteins, GRP 1-14 and HRG 1-9, the position of ligand insertion and any additional modifications are indicated. GRP chimeric envelope proteins (GRP 1-14) were generated by inserting the 21 amino acid GRP ligand into the Mlu I and Not 1 sites of previously constructed chimeric envelopes. The sequence encoding CAAAEQRLGNQWAVGHLMTRC SEQ ID NO:18) was generated by annealing two oligonucleotides: GRP_{A} GGCCGAGCAGCGCCTGGGCAACCAGTGGGCCGTCGGCCACCTGATGA; SEQ ID NO:19) and GRP_{B} (CGCGTCATCAGGTGGCCGACGGCCCACTGGTTGCCCAGGCGCTGCTC; SEQ ID NO:20). HRG chimeric envelope proteins (HRG 1-9) were generated by inserting a modified 49 amino acid binding region of the heregulin-β protein (Ballinger et al., 1998, J. Biol. Chem. 273:11675-11684) into the Mlu I and Not I sites of previously constructed chimeric envelopes. The 49 amino acid HRG sequence was derived by annealing four oligonucleotides: HRG_{A} (GGCCGCTTCACACCTTGTAAAGTGCGCAGAGAAGGAAAAGACGTTCTGC-GTCAACGGCGTGAGTGTTACAG; SEQ ID NO:21), HRG_{B} (GCCGTAGGTCTTAAC-CCTGTAACACTCACCGCCGTTGACGCAGAACGTCTTTTCCTTCTCTGCGCA CTTTACAAGGTGTGAAGC; SEQ ID NO:22), HRG_{C} (GGTTAAGACCTACGGCTATCTGATGTGCA-AGTGTCCGAACGAGTTCACGGGTGACCGGTGCCAGAACTACGTCATCG TCGA; SEQ ID NO:23), and HRGD (CGCGTCGACGCGATGACGTAGTTCTGGCACCGGTC-ACCCGTGAACTCGTTCGGACACTTGCACATCAGATA; SEQ ID NO:24). Experiments using the HRG chimeric envelope proteins are discussed below (Example 8).

TABLE 2
Description of GRP and HRG viruses

| | | |
|---|---|---|
| | Position of | |
| | Ligand | |
| | Insertion | Deletion of |
| | (A.A. | Nucleotides |
| ENV # | Location) | in Envelope |

| | |
|---|---|
| GRP CAAA-EQRLGNQWAVGHLM-TRC | (SEQ ID NO:18) |

| | | |
|---|---|---|
| GRP-1 | 1 | |
| GRP-2 | 38 | |
| GRP-3 | 38 | 5990-6082 |
| GRP-4 | 68 | |
| GRP-5 | 68 | 6082-1916 |
| GRP-6 | 120 | |
| GRP-7 | 120 | 6238-6281 |
| GRP-8 | 185 | |
| GRP-9 | 230 | |
| GRP-10 | 235 | |
| GRP-11 | 310 | |
| GRP-12 | 321 | |
| GRP-13 | 382 | |
| GRP-14 | 388 | |
| | Del. 3 A.A. | |
| | \| | |
| | FM DPSRYL | M |
| HRG CAAA- | | |

(SEQ ID NO:26)

SHLVKCAEKEKTFCVNGGECYRVKTYGYLMCKCP
NEFTGDRCQNYVIAS-TRC

| | | |
|---|---|---|
| HRG-1 | 1 | |
| HRG-2 | 38 | |
| HRG-3 | 38 | 5990-6082 |
| HRG-4 | 68 | |
| HRG-5 | 68 | 6082-1916 |
| HRG-6 | 120 | |
| HRG-7 | 185 | |
| HRG-8 | 230 | |
| HRG-9 | 235 | |

Pseudotyped virus producer cells were generated for each chimeric envelope derivative and the resultant GRP viruses were initially tested for transduction of host NIH 3T3 cells. Briefly, NIH 3T3 cells, human A375 melanoma cells, and human MDA-MB-231 breast carcinoma cells were infected with a GRP virus, selected with G418 for two weeks, fixed, stained with Giemsa and colonies counted. Amphotropic (Amph) and ecotropic viruses (Eco) were generated by expressing the wild type amphotropic and ecotropic envelopes, pCAA and pCEE, respectively. The amphotropic envelope, pCAA, and the LAPNL packaging vectors were generated as described herein and as is practiced in the art; the latter expresses the secreted alkaline phosphatase gene (SEAP) and the neomycin resistance gene. FIG. 4 (note the log scale) shows that all of the GRP viruses transduced NIH 3T3 cells except when the ligand was inserted within the N-terminal region (GRP-2, GRP-3) or in one case within the VRA (GRP-5). In general, the GRP viruses transduced NIH 3T3 cells with efficiencies comparable to that observed for RGD viruses.

A375 human melanoma and 231 breast carcinoma cells overexpress the GRP receptor (Yano et al., 1992, Cancer Res. 52:4545-4547; Pansky et al., 1997, Eur. J. Clin. Invest. 27:69-76; Miyazaki et al., 1998, Eur. J. Cancer 34:710-717). GRP viruses with insertions at the N-terminus (GRP-1), within the N-terminal region (GRP-2, GRP-3), within the VRA (GRP-4, GRP-5), downstream of the VRB (GRP-8) and upstream of the PRR (GRP-9) transduced both of these human cell lines. In contrast, GRP viruses with insertions within the PRR (GRP-10) or C-terminal region (GRP-11-GRP-14) failed to transduce human cells.

### Example 7

### Requirement for GRP Receptor Expression

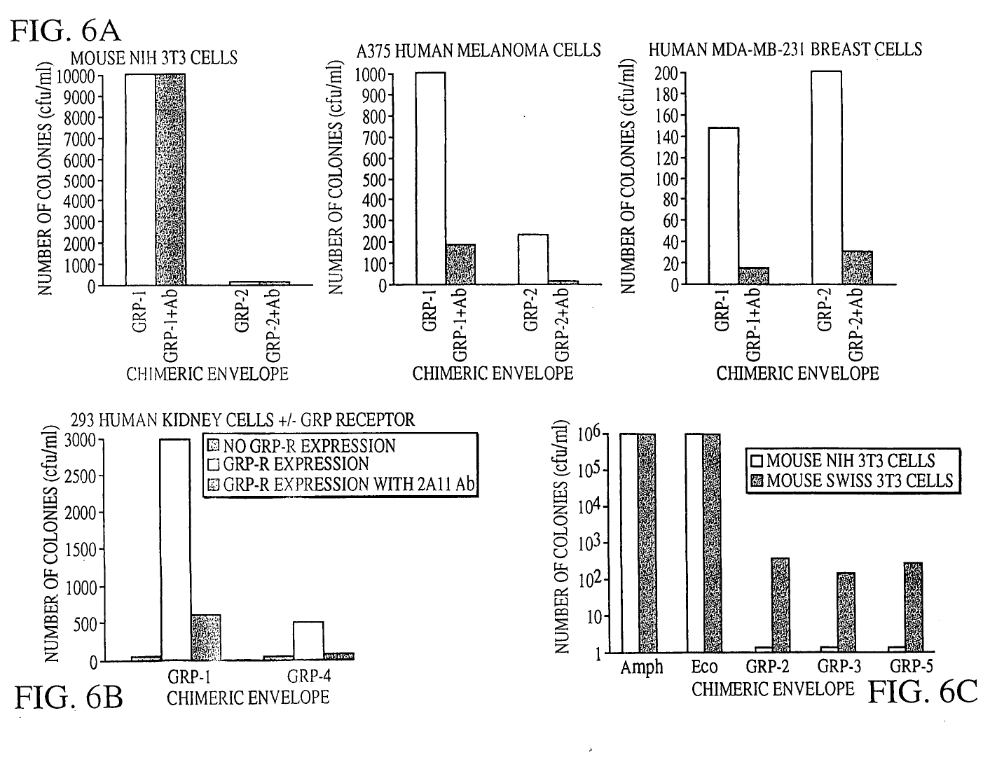

Experiments were performed to confirm that expression of the GRP receptor is required for GRP viruses to transduce human cells. First, it was tested whether treatment of GRP viruses with an antibody to the GRP protein would block transduction of human cells. GRP-1 or GRP-2 viruses were pretreated with 2A11 antibody (provided by Dr. Frank Cuttitta). NIH 3T3, A375 human melanoma cells, or MDA-MB-231 breast carcinoma cells were then infected with 2A11 antibody treated GRP or untreated virus and transduction analyzed as described above (see FIG. 4). The 2A11 antibody was added to pseudotyped virus at a 1:100 dilution followed by incubation at 4° C. for four hours and then viral infection was analyzed. FIG. 6A shows that 2A11, an antibody to the C-terminal region of GRP protein, substantially reduced transduction of both human cancer cell lines but not mouse NIH 3T3 cells. Thus, GRP is required for transduction of human but not mouse cells.

The question of whether expression of the GRP receptor is required for transduction of human cells by GRP viruses was examined. Human 293 cells do not express the GRP receptor (Valdenaire et al., 1998, FEBS Lett. 424:193-196). A 293 cell line was developed that constitutively expresses the GRP receptor (293-GRPR cells) using methods known in the art. Briefly, the GRPR-Zeo construct was generated by insertion of the GRP receptor gene (GRP—R) (provided by Dr. James F. Battey, NIH) into pcDNA3.1/Zeo+ (Invitrogen). 293-GRPR-Zeo cells were generated by transfection of 293 kidney cells with GRPR-Zeo, selection with Zeocin™, and verification of GRP receptor expression by RT/PCR. 293-GRPR-Zeo cells were infected with the GRP-1 or GRP-4 virus, with or without preincubation with the 2A11 antibody and transduction analyzed as described herein. FIGS. 6A and 6B show that 293-GRPR cells, but not the parental 293 cells, were transduced by GRP viruses and that pretreatment with the 2A11 antibody blocked transduction.

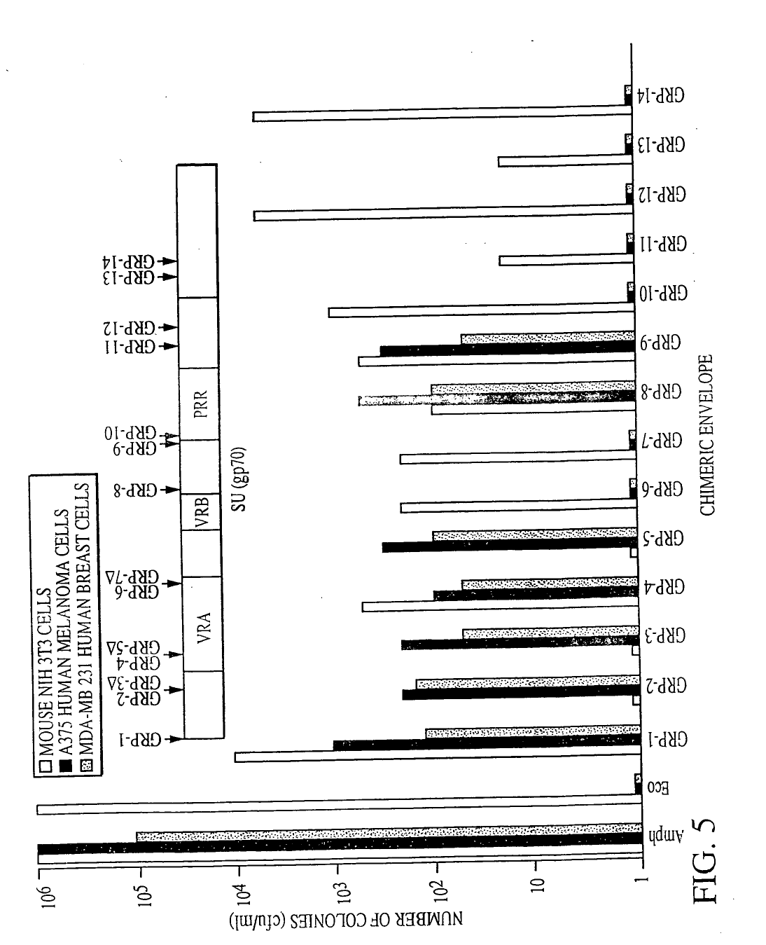

In a similar experiment, the requirement of the GRP receptor for transduction of mouse cells by GRP-2, GRP-3 and GRP-5 viruses was investigated. In these experiments, NIH 3T3 and Swiss 3T3 cells were infected with a GRP virus and transduction analyzed as described herein. FIG. 6C shows the results of these experiments. Several of the GRP viruses transduced mouse Swiss 3T3 cells, which express the GRP receptor, but not NIH 3T3 cells, which lack the GRP receptor. Collectively, the results shown in FIGS. 5 and 6 indicate that transduction of human cells by GRP viruses requires a virus bearing a chimeric GRP envelope derivative and a cell expressing a GRP receptor.

### Example 8

### Chimeric Envelope Proteins Containing HRG Heterologous Short Peptide Ligands

To test the ability of another heterologous short peptide ligand to alter retroviral tropism when inserted into an envelope protein, a series of chimeric envelope proteins containing the 56 amino acid heregulin-β peptide sequence (HRG; Table 2) were constructed. A polypeptide of residues 177 to 226 of HRG binds to and activates the HER3 and HER4 receptor, and was selected as the target ligand (Barbacci et al., 1995, J. Biol. Chem. 270:9585-9589). This ligand was modified through eleven substitutions known to increase its affinity for the homodimeric HER3 (Ballinger et al., 1998, J. Biol. Chem. 273:11675-11684; Table 2). Sequence encoding HRG ligand was inserted into MLV envelope gene locations that resulted in chimeric envelope proteins that had enabled transduction of human cells by GRP viruses and RGD viruses supra.

HRG viruses were first tested for their ability to transduce NIH 3T3 cells then MDA-MB-453 and MDA-MB-231 breast carcinoma cells were infected with an HRG virus and transduction analyzed as described herein. The transduction efficiencies of the HRG-8 and HRG-9 viruses were comparable to the equivalent GRP viruses (see GRP-9 and GRP-10; FIG. 4). By contrast, the transduction efficiencies of the HRG-1, HRG-4, HRG-6 and HRG-7 viruses were significantly lower than the equivalent GRP viruses (GRP-1, GRP-4, GRP-6, GRP-8).

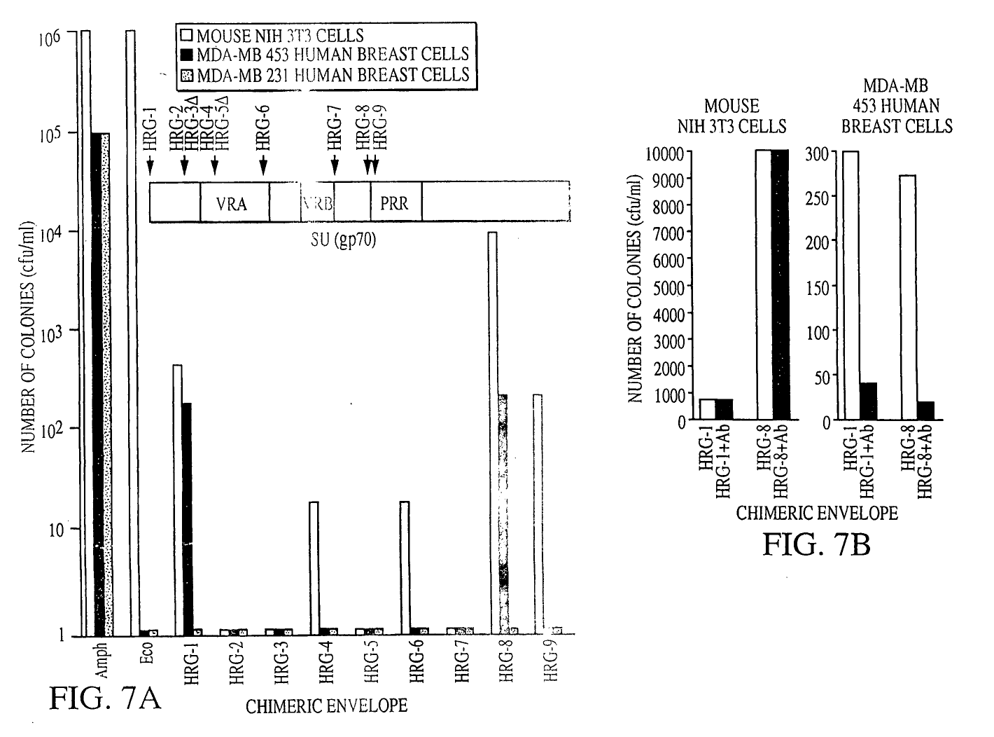

MDA-MB-453 breast carcinoma cells overexpress EGFR family members, whereas MDA-MB-231 breast carcinoma cells do not (Baulida and Carpenter, 1997, Exp. Cell Res. 232:167-172; Jeschke et al., 1995, Int. J. Cancer 60:730-739; Chan et al., 1995, J. Biol. Chem. 270:22608-22613). FIG. 7A shows that the HRG-1 and HRG-8 viruses transduced MDA-MB-453 but not MDA-MB-231 cells. The HRG-1 and HRG-8 viruses also transduced two other human breast cancer cell lines that overexpress EGFR family members: MCF-7 and AU-565 cells. In contrast, HRG-2, HRG-3, HRG-4, HRG-5, and HRG-7 failed to transduce MDA-MB-453 cells. This differs from the results with chimeric envelope proteins that have insertions of GRP peptide ligands in corresponding loci.

Experiments were conducted to test whether antibodies to HER-3 and HER-4 receptors block transduction of human cells by HRG viruses. In these experiments, NIH 3T3 and MDA-MB-453 breast carcinoma cells were pretreated with antibodies to HER-3 and HER-4 receptors (Lab Vision Corporation) and then infected with the HRG-1 or HRG-8 virus. Transduction was analyzed as described herein. Pretreatment of MDA-MB-453 cells with HER3 and HER4 antibodies substantially decreased transduction by HRG-1 and HRG-8 viruses indicating that viral entry was mediated by the HRG-receptor interaction (FIG. 7B).

### Example 9

### Use of Pseudotyped Viruses with Chimeric Envelope Proteins for Killing Cancer Cells

One use for viruses containing chimeric envelope proteins that redirect host specificity is for delivery of therapeutically useful genes to target cells such as cancer cells. Experiments were performed to test whether retroviruses bearing an appropriate chimeric envelope derivative can deliver a therapeutically useful gene to cancer cells. Mammalian cells expressing the herpes simplex virus thymidine kinase (TK) gene are killed by treatment with ganciclovir (Cheng et al., 1983, Proc. Natl. Acad. Sci. USA 80:2767-2770). The GRP-1 virus carrying the HSV TK gene was used to transduce A375 human melanoma and MDA-MB-231 breast carcinoma cells.

Briefly, A375 human melanoma cells and MDA-MD-231 human breast carcinoma cells were infected with GRP-1 virus expressing either the SEAP or TK gene. The packaging vector, LTKNL, containing the TK gene, was generated by removal of the SEAP gene from an LAPNL packaging vector and insertion of the thymidine kinase gene (TK; provided by Steve Jones, University of Massachusetts Medical School). GRP virus with the LTKNL packaging construct was generated and used to transduce human cells. Cells were selected with G418 for two weeks, followed by isolation of colonies and culture in media containing 10 μg/ml ganciclovir (Moravek Biochemicals, Inc.) and the cell densities were examined using a Zeiss Axiophot microscope.

Following ganciclovir treatment of transduced melanoma and breast carcinoma cells significant cell death was evident, whereas there was no cytopathic effect in ganciclovir treated cells transduced by a control GRP-1 virus not expressing the TK gene.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A chimeric retrovirus envelope protein comprising an ecotropic envelope protein and a heterologous short peptide ligand inserted within the ecotropic envelope protein.

2. The chimeric envelope protein of claim 1, wherein the ecotropic envelope protein is a Murine Leukemia Virus (MLV) envelope protein.

3. The chimeric envelope protein of claim 1, wherein the ecotropic envelope protein is a wild type envelope protein.

4. The chimeric envelope protein of claim 1, wherein the heterologous short peptide ligand is selected from the group consisting of an RGD ligand, a human epidermal growth factor receptor (HRG) ligand, or a gastrin releasing protein (GRP) ligand.

5. The chimeric envelope protein of claim 1, wherein the heterologous short peptide ligand is flanked by at least one cysteine on each side.

6. The chimeric envelope protein of claim 1, wherein the heterologous short peptide ligand is inserted into a conserved region of a wild-type envelope protein.

7. A nucleic acid molecule comprising a nucleic acid sequence encoding the recombinant chimeric envelope protein of claim 1.

8. A vector comprising a nucleic acid sequence encoding a chimeric envelope protein that contains a heterologous short peptide ligand.

9. The vector of claim 8, wherein the vector further comprises a nucleic acid sequence that encodes a therapeutically useful polypeptide.

10. A recombinant retroviral particle comprising a chimeric envelope protein comprising a heterologous short peptide ligand.

11. The recombinant retroviral particle of claim 10, wherein the retroviral particle can infect a mouse cell.

12. The recombinant retroviral particle of claim 10, wherein the retroviral particle cannot infect a mouse cell.

13. A method of altering retroviral tropism, the method comprising
(a) introducing into the genome of a retrovirus a nucleic acid sequence that encodes a chimeric envelope protein, and wherein
(b) the nucleic acid sequence of the chimeric envelope protein comprises a heterologous short peptide ligand, thereby producing a pseudovirus having altered tropism.

14. The method of claim 13, wherein murine leukemia virus (MLV) retroviral tropism is altered.

15. The method of claim 13, wherein the pseudovirus does not express wild-type envelope protein.

16. The method of claim 14, wherein the heterologous short peptide ligand is inserted into a conserved region of a wild-type envelope protein.

17. A method of identifying a nucleic acid sequence encoding a chimeric envelope protein that alters viral tropism, the method comprising
(a) introducing into the genome of a retrovirus, a nucleic acid sequence encoding a recombinant envelope protein comprising a heterologous short peptide ligand to produce a recombinant virus;
(b) infecting a target host cell with the virus; and
(c) assaying transduction of the target host cell by the virus, such that transduction of the host cell by the virus indicates that the nucleic acid sequence encodes a chimeric envelope protein that alters viral tropism.

18. The method of claim 17, wherein the virus is an MLV.

19. The method of claim 17, wherein the heterologous short peptide ligand is in a conserved region of the MLV envelope protein.

20. The method of claim 17, wherein the target host cell is a human cell.

21. The method of claim 17, wherein the target host cell is a cancer cell.

22. The method of claim 17, wherein the target host cell comprises a mutant gene and the retrovirus comprises a wild type nucleic acid sequence corresponding to the mutant gene.

23. The method of claim 17, wherein the chimeric envelope protein contains an RGD ligand, an HRG ligand, or a GRP ligand.

24. A method of delivering a nucleic acid sequence to a cell, the method comprising,
(a) providing a cell; and
(b) infecting a cell with a virus comprising a chimeric envelope protein and the nucleic acid sequence, wherein the chimeric envelope protein comprises a heterologous short peptide ligand.

25. The method of claim 24, wherein the heterologous short peptide ligand is an RGD ligand, an HRG ligand, or a GRP ligand.

26. The method of claim 24, wherein the cell is a mammalian cell.

27. The method of claim 24, wherein the cell is a human cell.

28. The method of claim 24, wherein the cell is a cancer cell.

29. The method of claim 24, wherein the cell is in an animal.

30. A method of treating cancer, the method comprising
(a) providing a cancer cell; and
(b) infecting a cancer cell with a virus, the virus comprising a chimeric envelope protein comprising a heterologous short peptide ligand and a therapeutically useful gene.

31. The method of claim 30, wherein the virus is a retrovirus.

32. The method of claim 30, wherein the cancer is in a mammal.

33. The method of claim 30, wherein the cancer is in a human.

34. The method of claim 30, wherein the therapeutically useful gene is encodes thymidine kinase.
